(19) United States
(12) Patent Application Publication
(10) Pub. No.: US 2006/0095045 A1
(43) Pub. Date: May 4, 2006

(54) Methods for explantation of intervertebral disc implants

(75) Inventor: Hai H. Trieu, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI Holdings, Inc.

(21) Appl. No.: 10/976,893

(22) Filed: Nov. 1, 2004

(51) Int. Cl. A61F 2/34

(52) U.S. Cl. 606/99

(57) ABSTRACT

Methods and devices are provided for the explantation of spinal implants. A cutting tool may be extended into the spinal implant. The spinal implant may be disintegrated into pieces and the pieces removed.

### FIELD OF THE INVENTION

The present invention relates to prosthetic spinal implants. More specifically, the present invention relates to methods and devices for explanting prosthetic spinal implants.

### DESCRIPTION OF THE RELATED ART

The intervertebral disc functions to stabilize the spine and to distribute forces between vertebral bodies. A normal disc includes a gelatinous nucleus pulposus, an annulus fibrosis and two vertebral end plates. The nucleus pulposus is surrounded and confined by the annulus fibrosis.

Intervertebral discs may be displaced or damaged due to trauma or disease. Disruption of the annulus fibrosis may allow the nucleus pulposus to protrude into the vertebral canal, a condition commonly referred to as a herniated or ruptured disc. The extruded nucleus pulposus may press on a spinal nerve, which may result in nerve damage, pain, numbness, muscle weakness and paralysis. Intervertebral discs also may deteriorate due to the normal aging process. As a disc dehydrates and hardens, the disc space height will be reduced, leading to instability of the spine, decreased mobility and pain.

One way to relieve the symptoms of these conditions is by surgical removal of a portion or the entire intervertebral disc. The removal of the damaged or unhealthy disc may allow the disc space to collapse, which would lead to instability of the spine, abnormal joint mechanics, nerve damage, and severe pain. Therefore, after removal of the disc, adjacent vertebrae are typically fused to preserve the disc space. Several devices exist to fill an intervertebral space following removal of all or part of the intervertebral disc in order to prevent disc space collapse and to promote fusion of adjacent vertebrae surrounding the disc space. Even though a certain degree of success with these devices has been achieved, full motion typically is never regained after such vertebral fusions. Attempts to overcome these problems have led to the development of disc replacement devices.

Disc replacement devices or intervertebral spinal disc implants or spinal implants are configured to be load bearing bodies of a size to be placed in an intervertebral disc space and intended to fully or partially replace the nucleus pulposus of mammals, particularly humans. Spinal disc implants are typically only prescribed when the natural nucleus pulposus becomes damaged or extruded.

Though replacement disc implant devices are available and generally work well for their prescribed use, they too may become damaged over time. In addition, prosthetic discs may be incorrectly sized for the intervertebral disc space that they occupy and therefore do not properly support the spinal column. This may lead to discomfort, pain, and other undesirable symptoms. To overcome this problem, the first prosthetic disc may need to be removed and replaced with a second prosthetic disc.

Spinal implants, especially those made from a gelatinous material such as a hydrogel, are typically implanted through a small defect or hole in the annulus fibrosis and are typically larger than the defect. For example, the implant may be inserted through a defect in the annulus fibrosis that initially allowed the natural nucleus pulposus to protrude. However, a defect in the annulus fibrosis that allows a natural nucleus pulposus to protrude also may allow a prosthetic spinal implant to protrude. Therefore, it is often favorable to keep any defect in the annulus fibrosis as small as possible. This is true when removing a natural nucleus pulposus and implanting or removing a prosthetic spinal implant.

U.S. Pat. No. 5,976,105 to Marcove (“the '105 patent”), U.S. Pat. Nos. 5,313,962 and 5,195,541 to Obenchain (“the '962 patent” and “the '541 patent,” respectively), and U.S. Pat. No. 4,678,459 to Onik (“the '459 patent”) all describe methods or instruments that relate to the removal of a natural nucleus pulposus. However, none of them relate to or disclose a method to remove a prosthetic spinal implant.

The '105 patent describes an intra-annular ultrasound disc apparatus and method. The patent aims to avoid unnecessary traumatization of the portions of the disc that are to be left intact. It further describes a method of inserting an ultrasonic probe inside the interior of the annular ligament, softening the tissue at the central region of the herniated disc, and inserting a discectomy instrument to remove the softened tissue.

Both the '962 patent and the '541 patent describe a method of performing laparoscopic lumbar discectomy, which is the excision, in part or whole, of an intervertebral disc. Specifically, both references describe penetrating the annulus and removing the herniated disc material.

Finally, the '459 patent discloses an irrigating, cutting, and aspirating system for percutaneous surgery. The patent further discloses a guillotine type cutting action to cut herniated disc tissue into small portions while the irrigation and vacuum means of the system aspirate the severed material. It also describes a means for cutting the nucleus pulposus of an intervertebral disc.

The cited references all describe means to remove a natural nucleus pulposus, typically using soft tissue shearing devices. In contrast to the natural nucleus pulposus, many spinal implants are hard polymeric plastic materials or even metal fusion cages. The soft tissue shearing devices used to remove the natural nucleus pulposus may be ineffectual in cutting the hard materials of a prosthetic implant. Other polymeric spinal implants are somewhat elastic, making them difficult to cut with conventional shearing devices. None of the disclosed methods of removing a nucleus pulposus, therefore, is entirely effective for removing a spinal implant.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

A need exists for a device and method to remove a spinal implant through a relatively small opening in the annulus fibrosis. Therefore, it is a feature of an embodiment of the present invention to provide for a method for explanting spinal implants using minimally invasive techniques. A retractable protective sleeve with an internal cutting tool may be guided to the spinal implant. The retractable protective sleeve may be retracted and the cutting tool projected into the spinal implant. The spinal implant may be disintegrated into pieces and the pieces removed.

In another embodiment, there is provided a device for explantation of a spinal implant. The device comprises a cutting tool inside a protective sleeve, a power source, and a handle to which the cutting tool, protective sleeve, and power source are attached.

These and other objects and advantages of the present invention will be apparent from the description provide herein.

### BRIEF DESCRIPTION OF THE FIGURES

FIG. 1 illustrates a side view of a cross-section of a nucleus pulposus implant in an intervertebral disc space, bound by a superior vertebral body, an inferior vertebral body, and an annulus fibrosis with a defect.

FIG. 2 illustrates intervertebral space of FIG. 1, with a cutting tool accessing the spinal implant through the annular defect.

FIG. 3 illustrates the intervertebral space of FIG. 2, with the cutting tool unsheathed and piercing the spinal implant.

FIG. 4 illustrates the intervertebral space of FIG. 3, with the cutting tool extending into varying depths of the intervertebral space and accessing the space through the annular defect at different angles. FIG. 4 further illustrates the implant of the previous Figures having been cut into pieces.

FIG. 5 shows the implant of the previous Figures, having been cut into many small pieces, being removed through the protective sleeve.

FIG. 6 illustrates a variety of cutting tips for a spinal implant explantation device and method of embodiments of the invention.

FIG. 7 illustrates preferred spinal implant explantation devices of embodiments of the invention.

FIG. 8 illustrates another preferred spinal implant explantation device of embodiments of the invention.

### DESCRIPTION OF THE PREFERRED EMBODIMENTS

The following description is intended to convey a thorough understanding of the present invention by providing a number of specific embodiments and details involving explantation of spinal implants. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

The terminology used herein is for the purpose of describing particular embodiments only and is not intended to limit the scope of the present invention. As used throughout this disclosure, the singular forms “a,” “an,” and “the” include plural reference unless the context clearly dictates otherwise. Thus, for example, a reference to “a spinal implant” includes a plurality of such implants, as well as a single implant, and a reference to “a cutting tool or probe” is a reference to one or more cutting tools or probes and equivalents thereof known to those skilled in the art, and so forth.

Unless defined otherwise, all technical and scientific terms used herein have the same meanings as commonly understood by one of ordinary skill in the art to which this invention belongs. All publications mentioned herein are cited for the purpose of describing and disclosing the various spinal implants, methods of explanting natural nucleus pulposus, and other components that are reported in the publications and that might be used in connection with the invention. Nothing herein is to be construed as an admission that the invention is not entitled to antedate such disclosures by virtue of prior invention.

Throughout this description, the expressions “natural nucleus pulposus” refers to a nucleus pulposus that is naturally found in the intervertebral disc space of a mammal, particularly humans. The expression is used to differentiate between what is a natural, normal body part and that which is a man-made implant.

The terms “spinal implant” or “nucleus implant” shall be used to denote any man-made implant which is used to partially or fully replace the natural nucleus pulposus or intervertebral disc that is found in mammals, especially humans. Man-made spinal implants include implants made from natural sources (e.g. implanted autologous bones and tissues), implants made from synthetic sources (e.g. metals, polymers, and ceramics), and composites thereof (e.g. bone/polymer matrices).

Spinal implants can be made of a wide range of materials such as polymeric materials, metals, ceramics, and body tissues. Exemplary polymeric materials include, but are not limited to, thermoplastic polymers, thermoset polymers, elastomers, hydrogels, adhesives, sealants, and composites thereof. Polymeric spinal implants may be preformed implants, injectable/in situ formable implants, and combinations thereof. Preformed polymeric spinal implants may be in any shape, including implants shaped like a spiral, hockey puck, kidney, capsule, rectangular block, cylinder, implants such as those described in, for example, U.S. Pat. No. 6,620,196, the disclosure of which is incorporated herein by reference in its entirety, and the like. Spinal implants, especially polymeric implants, also may comprise supporting bands or jackets.

Spinal implants may be in any of numerous known forms, including, but not limited to, total disc prostheses, intervertebral fusion devices, stackable corpectomy devices, threaded fusion cages, and impacted fusion cages. Spinal implants also include implants wherein only the full or partial nucleus of the intervertebral disc is replaced, for example nucleus replacement implants and nucleus augmentation implants. Because the invention is adept at removing a spinal implant through a small defect in the annulus fibrosis, it is preferred that the spinal implant be a nucleus replacement implant or nucleus augmentation implant wherein the natural annulus fibrosis is retained.

Exemplary implants include hydrogel implants that are injected into an evacuated disc space. The implant hardens into a implant shaped like the evacuated disc space. Such implants may be removed at a later time through practice of the present invention if they are damaged or to replace them with better functioning implants, such as preformed implants like the Nautilus®.

The phrase “opening in the annulus fibrosis” shall denote any opening, hole, or other defect in the annulus fibrosis. It is through an opening in the annulus fibrosis that the spinal implant preferably is removed. The opening in the annulus fibrosis preferably is less than about 20 mm in the largest dimension, and may be comprised of any shape, such an ellipse, circle, square, etc. In a more preferred embodiment, the opening in the annulus fibrosis preferably is less than 15 mm in the largest dimension. In a most preferred embodiment, the opening in the annulus fibrosis is less than 10 mm in the largest dimension. Because the invention provides for removal of spinal implants through small openings in the annulus fibrosis, the patient's natural annulus fibrosis preferably may be uninjured during the explantation procedure and may be retained after implant explantation.

“Disc space” means the volume occupied, or formerly occupied, by the spinal implant. The disc space may be the volume contained inside the annulus fibrosis. The disc space also may be the entire volume, including the annulus fibrosis, between two adjacent vertebral bodies.

An embodiment of the present invention provides a device for explantation of a spinal implant. The device may be referred to as an “explantation instrument.” The explantation instrument may comprise a cutting tool, a protective sleeve, a power source, and a handle to which the cutting tool, protective sleeve, and power source are attached.

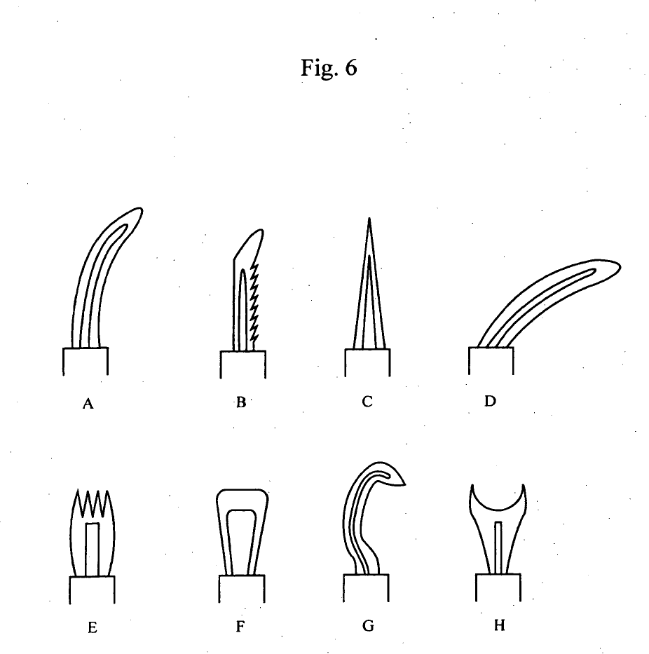

The cutting tool may comprise a mechanical cutting element. The mechanical cutting element preferably is located at the tip of the cutting tool. The mechanical cutting element may comprise, for example, a flat blade, curved blade, saw blade, pointed probe, angle blade, saw tip, knife tip, hook tip, or C-tip. Exemplary mechanical cutting elements are illustrated in FIG. 6. Embodiment A illustrates a curved blade; embodiment B illustrates a saw blade; embodiment C illustrates a pointed probe; embodiment D illustrates an angle blade; embodiment E illustrates a saw tip; embodiment F illustrates a knife tip; embodiment G illustrates a hook tip; and embodiment H illustrates a C-tip. In other embodiments, the mechanical cutting element may comprise a drill bit.

One skilled in the art will appreciate the various configurations that the mechanical cutting element may take, and all such configurations and modifications thereof are considered within the scope of the invention. For example, the mechanical cutting elements may come in various sizes, lengths, thicknesses, shapes, and so forth. Preferably, the mechanical cutting element is sufficiently rigid to as to effect penetration and cutting of a spinal implant. In a preferred embodiment, the mechanical cutting element also is detachable and disposable so that the mechanical cutting element may be replaced with a new, sterile mechanical cutting element following an explantation procedure.

In a preferred embodiment, the explantation instrument may additionally comprise mechanical means to gyrate, rotate, oscillate, or reverberate the mechanical cutting element. For example, if the mechanical cutting element is a saw blade, it may be preferred that the explantation instrument additionally comprise mechanical means to oscillate the saw blade back and forth so as to effect cutting of the spinal implant. Alternatively, the various knife tips also can be oscillated back and forth to effect cutting of the spinal implant or even rotated about their axis like a drill bit. One skilled in the art will appreciate the various mechanical means, for example electric motors and gear arrangements, that may be used to effect gyration, rotation, oscillation, reverberation, and so forth of the mechanical cutting element. Preferably, the mechanical means may be continuously adjusted between an off state and full power so as to control the gyration, rotation, oscillation, reverberation, and so forth of the mechanical cutting element.

The cutting tool may additionally comprise a heating element. The heating element preferably is located at the tip of the cutting tool. Any applicable source of thermal energy may be used as the heating element. The heating element may heat the spinal implant directly or may heat the mechanical cutting tool. Exemplary heating elements include, but are not limited to, electric resistance heaters, sources of ultrasonic vibrations, and lasers. For example, the mechanical cutting element itself may be an electric resistance heater wherein electric current passes through the mechanical cutting element. In another embodiment, an electric heating element, for example a thin metallic wire, may be embedded in the mechanical cutting element. This is exemplarily illustrated in FIG. 6, embodiments A-H, where wire leads acting as heating elements are shown running through the exemplary mechanical cutting elements. In another embodiment, a source of laser energy may be disposed immediately adjacent to the mechanical cutting element of the cutting tool.

In a preferred embodiment, the heating element heats the mechanical cutting element to at least 100° C. In a more preferred embodiment, the heating element heats the mechanical cutting element to at least 150° C. In a most preferred embodiment, the heating element heats the mechanical cutting element to greater than 200° C. The temperature of the heating element may preferably be continuously adjusted between an off state and full power. Heating elements such as the exemplary heating elements described herein may be desirable to soften the spinal implant, thereby facilitating faster and easier disintegration of the spinal implant. Heating elements may be especially preferred when the spinal implants are made of polymeric materials that will soften relatively quickly in response to elevated temperature.

The cutting tool preferably may be adjustable to facilitate disintegration of the spinal implant. For example, the cutting tool may be bendable so that the tool can curve. This may be preferable because a spinal implant may be irregularly shaped and a bendable cutting tool is more likely to be able to reach all parts of the irregularly shaped spinal implant. The cutting tool also preferably may be steerable to that the user may direct the cutting tool to that portion of the spinal implant that is to be disintegrated. The cutting tool also may preferably be extensible. One skilled in the art will appreciate other ways in which the cutting tool preferably may be adjustable in order to facilitate disintegration of the spinal implant.

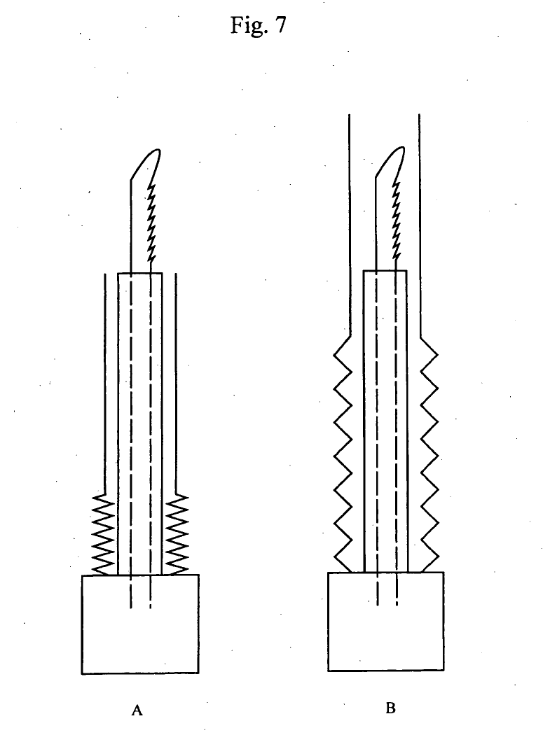

A protective sleeve may surround the cutting tool in order to prevent unwanted contact between the cutting tool and tissues that are not to be excised or otherwise damaged during explantation of the spinal implant. The protective sleeve may be retractable so that, when desired, the protective sleeve may be retracted, thereby projecting the cutting tool into adjacent tissues and structures, such as the spinal implant. Additionally, the protective sleeve may be extensible so that, when desired, the protective sleeve again may be extended beyond the cutting tool, thereby shielding adjacent tissues and structures from the cutting tool. In this way, the cutting tool may be preferentially exposed for use in excision of tissue and explantation of the spinal implant. FIG. 7 illustrates an exemplary protective sleeve. Embodiment A illustrates the protective sleeve in a retracted position, exposing the cutting tool. Embodiment B illustrates the protective sleeve in an extended position, shielding the cutting tool.

In a preferred embodiment, the protective sleeve is electrically and thermally insulated. Electrical insulation may be desirable to prevent unwanted stray of the electrical current from the heating element. Additionally, electrical insulation is a safety feature in general to prevent unwanted electrical discharge from the device as a whole. Thermal insulation may be desirable to protect tissues and structures adjacent to the cutting tool from damage incurred due to heat radiated by the optional heating element. The protective sleeve may be made from any applicable polymeric, ceramic, metallic, and composite materials so as to achieve desirable thermal and electrical insulative qualities.

The protective sleeve may be detachable and disposable. A detachable protective sleeve may be desirable so that, upon explantation of the spinal implant, the sleeve may be detached from the rest of the explantation instrument. For example, the sleeve may be left in the body and the remainder of the explantation instrument may be removed. The sleeve then may function as a cannula for removal of the pieces of the spinal implant. Additionally, a detachable sleeve may thereby be disposable, so that a new, sterile sleeve may be used in subsequent procedures involving the explantation instrument. The protective sleeve, like the cutting tool, also preferably may be adjustable in that it may be bendable, extensible, and steerable. This may aid in directing the protective sleeve to the spinal implant through the tissues, vasculature, and structures of the body. Also, a bendable, extensible, and steerable protective sleeve may be preferable so that the sleeve may be steered inside the disk space during removal of the pieces of the spinal implant, for example by vacuum and irrigation.

In a preferred embodiment, a flexible scope or camera may be attached to the end of the protective sleeve. The scope or camera may be desirable to enable the user to more easily steer the protective sleeve and cutting tool to the spinal implant.

The power source may be any applicable source of electrical energy. In a preferred embodiment, the power source is a battery. The battery may preferably be encased in the handle of the explantation instrument. The battery also may preferably be rechargeable so that it can be reused after the electrical capacitance of the battery is discharged. The battery may be any applicable type of battery, including, but not limited to, lithium batteries, fuel cells, nickel-cadmium batteries, and the like. It may be preferred that the battery, especially if it is not rechargeable, be removable so that the battery may be replaced with a new battery after it has been discharged. If the battery is rechargeable, it may still be preferred that the battery be removable so that it may be recharged in an external charger separate from the explantation instrument itself. One skilled in the art will appreciate the various configurations that the battery and other power sources may take, in accordance with the limitations herein.

The handle may be any applicable means for holding the explantation instrument. One skilled in the art will appreciate the various applicable configurations that the handle may take, including finger grips, various shapes, triggers to operate the explantation instrument, clips to attach other surgical tools and instruments, surface textures to ensure a good grip, and the like. All such configurations and modifications are understood to be within the scope of the invention. Preferably, the handle may include adjustable switches to control the temperature of the heating element and the mechanical actuation of the mechanical cutting element. In a preferred embodiment, the handle may include detachment means whereby the cutting tool and protective sleeve may be detachably connected to the handle of the explantation instrument. One skilled in the art will appreciate how this is to be done. If the explantation instrument comprises mechanical means to actuate the mechanical cutting means, it may be preferable that a portion of the means be located inside the handle.

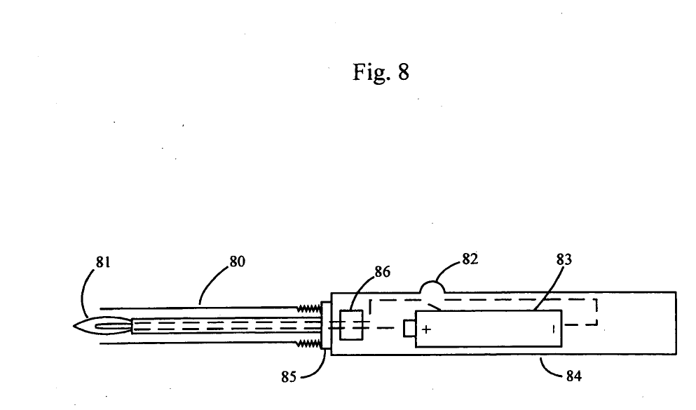

FIG. 8 exemplarily illustrates a device for explantation of a spinal implant in accordance with the invention. The device comprises a cutting tool 81. The cutting tool comprises a mechanical cutting element and a heating element. Mechanical means 86 may gyrate, rotate, oscillate, or reverberate the mechanical cutting element. The cutting tool is internal to a protective sleeve 80 that may be preferentially extended and retracted to protect and expose the cutting tool. Detachment means 85 detachably connect the cutting tool and protective sleeve to the handle 84 of the instrument. The power source is a battery 83 that may be operated with a switch 82 to control the delivery of power to the heating element of the cutting tool 81 and mechanical means 86 to gyrate, rotate, oscillate, or reverberate the mechanical cutting element.

In another embodiment, the protective sleeve surrounding the cutting tool is guided to the spinal implant. The protective sleeve preferably may be extensible so that it may be elongated while being guided to the spinal implant. Guiding to the spinal implant may be accomplished by manipulating the handle of the explantation instrument to steer the protective sleeve and cutting tool to a position immediately adjacent to the spinal implant. The optional scope or camera preferably may aid in this process. The protective sleeve may be retracted to expose the cutting tool. The cutting tool may be projected into the spinal implant and manipulated so as to disintegrate the spinal implant. The optional mechanical means may aid in this process by causing the mechanical cutting element to gyrate, rotate, oscillate, or reverberate in such a manner as to facilitate disintegration of the spinal implant.

The cutting tool may disintegrate the spinal implant into pieces by cutting the spinal implant, melting the spinal implant, or a combination thereof. In this way, the spinal implant may be separated into smaller pieces that then may be more easily removed from the space formerly occupied by the spinal implant. When the spinal implant is satisfactorily disintegrated, the protective sleeve may be extended and the cutting tool retracted so as to again surround the cutting tool. In a preferred embodiment, the protective sleeve then may be detached from the explantation instrument, including the cutting tool. In a more preferred embodiment, the protective sleeve then may be allowed to remain in the body while the rest of the explantation instrument is removed. In this way, the protective sleeve will continue to afford access to the disc space without the obstruction of the internal cutting tool.

The pieces of the spinal implant may be removed from the space formerly occupied by the spinal implant in any applicable manner, as will be appreciated by one skilled in the art. For example, the pieces of the spinal implant may be removed by irrigating the disc space with water or saline solution. An irrigation solution may be supplied to the disc space through the protective sleeve. Alternatively, the irrigation solution may be supplied to the disc space through a separate cannula that is inserted to replace or in addition to the protective sleeve. Pieces of the spinal implant also may be removed by vacuuming the pieces of the spinal implant out of the disc space. Vacuum may be applied through the protective sleeve or a cannula inserted to replace or in addition to the protective sleeve. Pieces of the spinal implant also may be removed using tweezers, forceps, a pituitary ronguer, or other surgical tools as will be appreciated by one skilled in the art. This may be preferable for larger pieces that are more difficult to extract, for example through the opening in the annulus fibrosis.

In a preferred embodiment, the cutting tool may be projected into the spinal implant through an opening in the annulus fibrosis. The spinal implant may be disintegrated into pieces smaller than the opening in the annulus fibrosis in order to facilitate easier removal of the spinal implant. In this way, a spinal implant may be removed without undue damage to the annulus fibrosis. In another preferred embodiment, the opening in the annulus fibrosis is not enlarged during explantation of the spinal implant.

In a more preferred embodiment, the opening in the annulus fibrosis through which the implant is to be removed was created prior to the explantation of the implant. For example, the opening in the annulus fibrosis may be created during implantation of the spinal implant. Rather than creating a new opening and further damaging the annulus fibrosis, the existing opening may be utilized to explant the spinal implant. Insertion of the cutting tool and removal of the implant pieces through an opening in the annulus fibrosis is especially preferred when the implant to be explanted is a nucleus replacement implant or nucleus augmentation implant. In this way, the annulus fibrosis retained during implantation of the spinal implant may not be further damaged during explantation of the spinal implant.

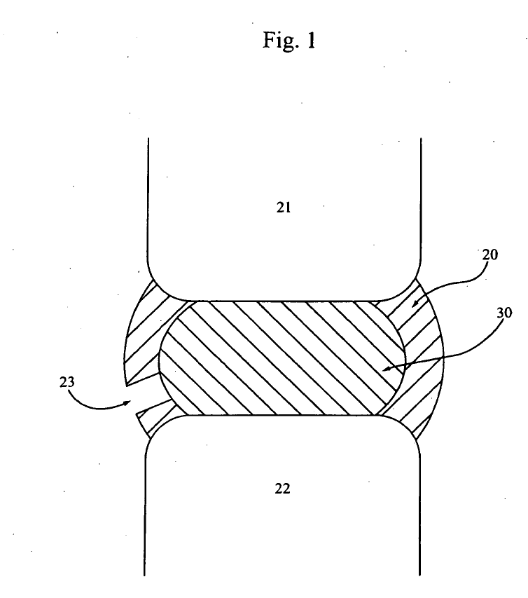

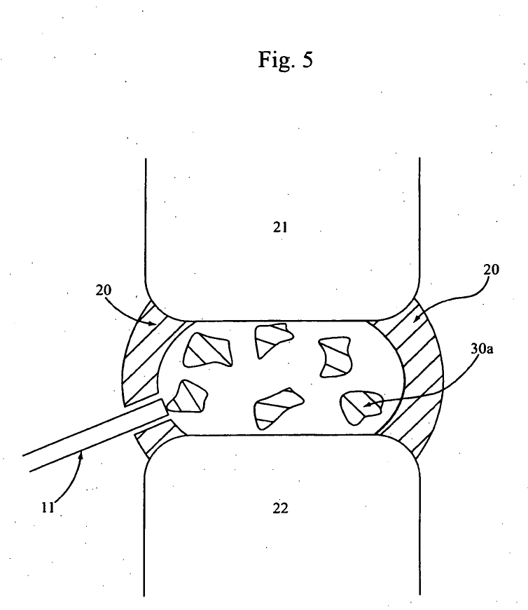

Embodiments of the invention will now be described in reference to FIGS. 1 to 5.

FIG. 1 illustrates a nucleus implant 30 between a superior vertebral body 21 and an inferior vertebral body 22. Preferably, the nucleus implant 30 is at least partially surrounded by the annulus fibrosis 20. The superior vertebral body 21, inferior vertebral body 22, and annulus fibrosis 20 define the boundaries of the intervertebral disc space that the implant 30 at least partially occupies. It is also preferable that the annulus fibrosis 20 has a defect or hole 23. It is further preferred that the defect 23 is a pre-existing condition, and was not caused by the performance of the present invention. Implant 30 also is preferably undersized, oversized, or damaged in some way and needs to be replaced. Throughout the description, the term “undersized” denotes that the implant is too small to properly support the axial loads of, or properly align the spinal column. Also throughout the description, the term “oversized” denotes that the implant is too large to properly support the axial loads of, or properly align the spinal column.

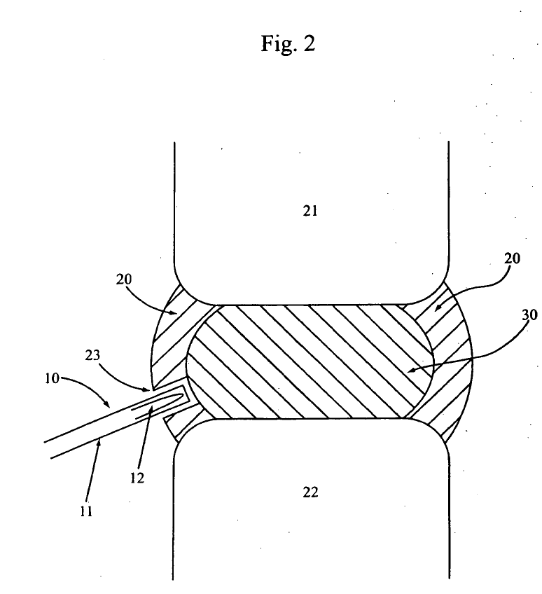

FIGS. 2, 3, and 4 depict a preferred embodiment of the invention that provides a probe 10 comprising a protective sleeve 11 housing a cutting tool 12 for insertion into a defect or hole 23 in the annulus fibrosis 20. The cutting tool 12 preferably comprises a heating element to melt, cut, and break down the implant material. Heated tips may be particularly effective when explanting a nucleus implant comprising elastic polymeric or thermoplastic materials, such as silicone-polyurethane based implants. The heat may be supplied by electric current, ultrasonic vibrations, laser energy, or other means known in the art. The cutting tool 12 also may preferably comprise a mechanical cutting element like a knife, a pointed tip like a needle, a blunt probe, or a reciprocating saw blade. Mechanical shearing without heat, such as with a knife edge or a reciprocating saw blade, also may be used, though mechanical shearing without heat may not be preferred if the spinal implant comprises elastic polymeric materials. In addition, the protective sleeve 11 preferably is insulated to protect the surrounding tissues and structures from being damaged by heat radiated from the heated cutting tool 12.

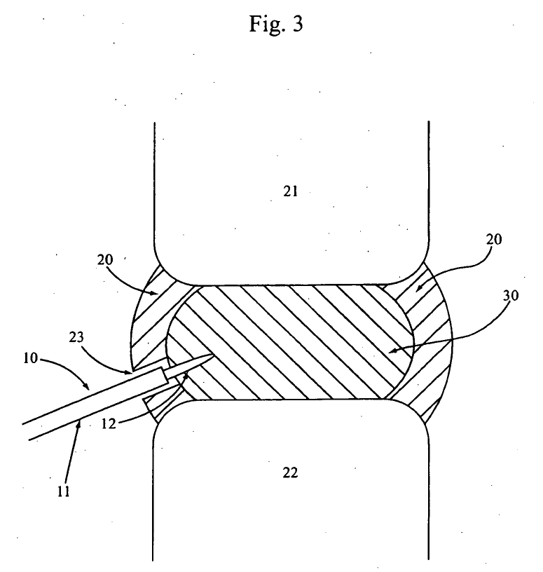

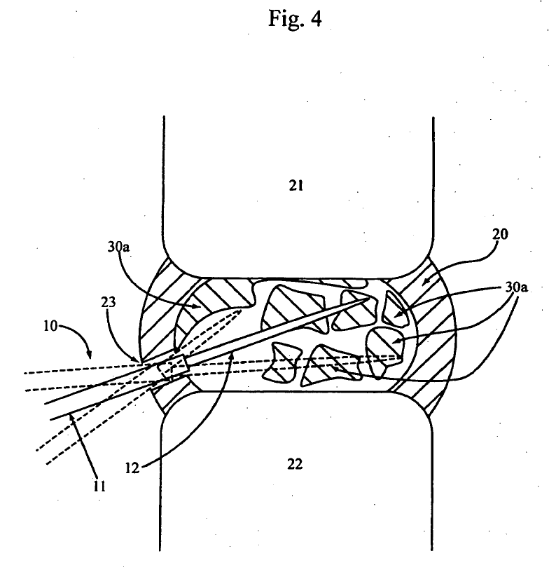

The probe 10 is guided through surrounding tissues and into the annular defect 23. Minimally invasive techniques to access the intervertebral disc space can be readily determined by those of ordinary skill in the art without undue experimentation. For example, fluoroscopic guidance may be used with the METRx® MicroDiscectomy System available from Medtronic Sofamor Danek. Once the probe 10 has reached the spinal implant 30, the protective sleeve 11 preferably is retracted and the cutting tool 12 preferably is extended into the intervertebral disc space and into the spinal implant 30, as illustrated in FIG. 3. Once inside the intervertebral disc space, the cutting tool 12 can be extended to varying depths and adjusted through varying angles about the annular defect 23 to disintegrate the spinal implant 30 into pieces 30a, as illustrated in FIG. 4.

Finally, after the implant 30 has been cut into sufficiently small pieces, the pieces 30a are removed. It is preferred that a vacuum is applied through the protective sleeve 11 to assist in removing the implant pieces 30a. The implant pieces 30a then are preferably removed by suction through the protective sleeve 11. It is also envisioned that the protective sleeve may be irrigated, thereby assisting in removing the implant pieces. The particular amount of vacuum and irrigation necessary to remove the implant pieces 30a can be easily determined by one of ordinary skill in the art without undue experimentation.

The foregoing detailed description is provided to describe the invention in detail, and is not intended to limit the invention. Those skilled in the art will appreciate that various modifications may be made to the invention without departing significantly from the spirit and scope thereof.

What is claimed is:

1. A method for explanting spinal implants, comprising:
inserting a cutting tool through an opening in the annulus fibrosis;
disintegrating the implant into pieces smaller than the opening in the annulus fibrosis; and
removing the pieces through the opening in the annulus fibrosis.

2. The method of claim 1, wherein the opening in the annulus fibrosis is a pre-existing condition that is not created through the exercise of the method.

3. The method of claim 1, wherein the opening in the annulus fibrosis is not enlarged while inserting the cutting tool, disintegrating the implant, and removing the pieces.

4. The method of claim 1, wherein the cutting tool is guided to the opening in the annulus fibrosis inside a protective sleeve.

5. The method of claim 4, wherein the protective sleeve is thermally and electrically insulated.

6. The method of claim 4, wherein the protective sleeve is extensible and retractable.

7. The method of claim 1, wherein the cutting tool comprises a mechanical cutting element.

8. The method of claim 7, wherein the mechanical cutting element comprises a flat blade, curved blade, saw blade, pointed probe, angle blade, saw tip, knife tip, hook tip, or C-tip.

9. The method of claim 1, wherein the cutting tool comprises a heating element.

10. The method of claim 9, wherein the heating element is an electric resistance heater, a source of ultrasonic vibrations, or a laser.

11. The method of claim 1, wherein removing the pieces comprises irrigating, vacuuming, or a combination thereof.

12. A method for explanting spinal implants, comprising:
guiding a retractable protective sleeve and internal cutting tool to the spinal implant;
retracting the protective sleeve;
projecting the cutting tool into the spinal implant;
disintegrating the spinal implant into pieces using the cutting tool; and
removing the pieces.

13. The method of claim 12, wherein the cutting tool is projected into the spinal implant through an opening in the annulus fibrosis.

14. The method of claim 13, where the opening in the annulus fibrosis is a pre-existing condition that is not created through the exercise of the method.

15. The method of claim 13, wherein the opening in the annulus fibrosis is not enlarged while guiding the retractable protective sleeve, retracting the protective sleeve, projecting the cutting tool, disintegrating the spinal implant, and removing the pieces.

16. The method of claim 12, wherein the protective sleeve is thermally and electrically insulated.

17. The method of claim 12, wherein the cutting tool comprises a heating element.

18. The method of claim 17, wherein the heating element is an electric resistance heater, a source of ultrasonic vibrations, or a laser.

19. The method of claim 12, wherein the cutting tool comprises a mechanical cutting element.

20. The method of claim 19, wherein the mechanical cutting element comprises a flat blade, curved blade, saw blade, pointed probe, angle blade, saw tip, knife tip, hook tip, or C-tip.

21. The method of claim 12, wherein disintegrating the spinal implant into pieces comprises cutting the spinal implant, melting the spinal implant, or a combination thereof.

22. The method of claim 12, wherein removing the pieces comprises irrigating, vacuuming, or a combination thereof.

23. The method of claim 12, wherein the pieces are removed through a defect in the annulus fibrosis.

24. A spinal implant explantation device, comprising:
a protective sleeve;
a cutting tool inside the protective sleeve;
a power source; and
a handle to which the cutting tool, protective sleeve, and power source are attached.

25. The device of claim 24, wherein the cutting tool comprises a heating element.

26. The device of claim 25, wherein the heating element is an electric resistance heater, a source of ultrasonic vibrations, or a laser.

27. The device of claim 24, wherein the cutting tool comprises a mechanical cutting element.

28. The device of claim 27, wherein the mechanical cutting element comprises a flat blade, curved blade, saw blade, pointed probe, angle blade, saw tip, knife tip, hook tip, or C-tip.

29. The device of claim 27, wherein the cutting tool comprises mechanical means to gyrate, rotate, oscillate, or reverberate the mechanical cutting element.

30. The device of claim 24, wherein the protective sleeve is separable and disposable.

31. The device of claim 24, wherein the protective sleeve is retractable and extensible.

32. The device of claim 24, wherein the cutting element is separable and disposable.

33. The device of claim 24, wherein the power source is a battery.

34. The device of claim 28, wherein the battery is encased inside the handle.
